(19) United States
(12) Patent Application Publication
(10) Pub. No.: US 2012/0253349 A1
(43) Pub. Date: Oct. 4, 2012

(54) CANNULATED SACRAL INTRODUCER RASP DEVICE

(76) Inventor: Gregory Flynn, Tampa, FL (US)

(21) Appl. No.: 13/074,572

(22) Filed: Mar. 29, 2011

(51) Int. Cl. A61B 17/16

(52) U.S. Cl. 606/85

(57) ABSTRACT

A wire-guided system or device for increasing the size of an opening at the base of the spine during the performance of surgery such as back surgery. The device has barbs for increasing the size of the opening and a channel for accepting a typical guide wire. The channel reduces the need to remove and reinsert the guide wire.

### FIELD

This invention relates to a wire-guided system or device for increasing the size of an opening during the performance of back surgery.

### BACKGROUND

Less invasive or “minimally invasive” surgical techniques have become increasingly popular, as physicians, patients and medical device innovators seek to reduce the trauma, recovery time and side effects typically associated with conventional surgery. The art of such less invasive surgical methods and devices has many challenges. For example, less invasive techniques involve working in a smaller operating field, working with smaller devices, and trying to operate with reduced or even no direct visualization of the structures being treated. These challenges are often compounded when target tissues of a given procedure reside very close to one or more vital, non-target tissues.

Many areas of surgery have moved from the traditional operating procedures to less invasive procedures. For example, in many cases, a gallbladder is removed through a tiny incision.

One area of surgery that has benefited from less invasive techniques is the treatment of spinal stenosis. Spinal stenosis occurs when nerve tissue and/or the blood vessels supplying nerve tissue in the spine become infringed upon by one or more structures in the lower spine leading to pain, numbness and/or loss of certain functions.

In the United States, spinal stenosis is frequent in adults aged 50 and older and is the most frequent reason cited for back surgery in patients aged 60 and older. Often, due to their weight and unsymmetrical weight characteristics, obese people are more apt to suffer from spinal stenosis.

Patients suffering from spinal stenosis are often treated with exercise therapy, analgesics, anti-inflammatory medications, and epidural steroid injections. When these conservative treatments do not work or the patient's symptoms are severe, surgery may be required to remove the infringing tissue and decompress the impinged nerve tissue.

Lasers have proven themselves incredibly valuable in lumbar spinal stenosis surgery. Prior to the use of lasers, an incision was made in the back, and muscles and supporting structures were stripped away from the spine to expose the vertebral column. Complete or partial removal of any bony arch covering the back of the spinal canal may then be performed. In addition, the surgery often includes partial or complete removal of all or part of one or more facet joints to remove infringing ligamentum flavum or bone tissue. Such spinal stenosis surgery was performed under general anesthesia and the patients required a five to seven day hospital stay, with full recovery taking between several weeks to three months. Therapy at a rehabilitation facility was often required to regain desired mobility.

Less invasive surgical methods and devices for treating spinal stenosis and other back problems often utilize a laser to remove the infringing tissue. “Epiduroscopy” by G. Schültze describes methods of performing spinal endoscopy using lasers. In this, G. Schültze describes methods for entering the epidural space, guiding a fiber optic probe into the epidural space with the help of a C-arm device and correcting various situations using the laser. In chapter 7.5, G. Schültze discusses the Epidural laser adhesiolysis, for example, using a 1064-nm Nd, YAG 1320-nm nd and a 940-nm laser for “coagulation of bleeding, rechanneling stenosis caused by tumors and destroying plaques in vessel walls.” In this, a fiber optic is introduced into the epidural space via a working channel of an epiduroscope under epiduroscope vision. A laser diode of from 1 watt to 25 watts fires a burst of energy through the fiber and onto the target tissue. G. Schültze describes that the light energy penetrates the tissues but is not significantly absorbed by the surrounding hemoglobin, melanin or water.

It is well known that different light frequencies are absorbed differently by different target materials. The described procedure uses lasers with a wavelength of from around 940-nm to 1320-nm. These wavelengths are selected because they are well absorbed by both hemoglobin and water, which are both major components of cartilage and scar tissue.

In another example of the prior art, a 532-nm (Green-light) laser has proven successful in treatment of the prostate and other urological conditions. A method referred to as Photo-Selective Vaporization has been successfully used on Benign Prostatic Hyperplasia (BPH) to remove enlarged prostate tissue, resulting in an open channel for urine flow. This specific wavelength of laser energy is selected because it is maximally absorbed by hemoglobin and, therefore, absorbed by tissue that has blood in it such as prostate tissue.

U.S. Pat. Pub. 2008/0267814 to Bornstein shows the value of multiple wavelength lasers for use in elimination of microbes. In this application, two wavelengths can include emission in two ranges approximating 850 nm to 900 nm and 905 nm to 945 nm at the same time. This application does not alternate the use the lasers depending upon the type of target tissue and not in the epidural space or spinal canal.

U.S. Pat. Pub. 2008/0103504 to Schmitz, et al, describes a method of removing ligamentum flavum tissue in the spine to treat spinal stenosis. There is no disclosure of the wavelength of laser or having multiple laser wavelengths.

U.S. Pat. Pub. 2008/0039828 to Jimenez, et al, describes using a laser of a particular wavelength specifically tuned to a biocompatible colorant. The target tissue is colored by the colorant and the laser used to vaporize the tissue that has been colored by the colorant. This disclosure describes a single laser of a wavelength that is absorbed by the colorant and, therefore, the target tissue is changed (in color) to better absorb the light energy of the fixed-wavelength laser.

During spinal endoscopy using lasers, the patient is positioned in the “prone” posture, providing access to the epidural area. To gain access to the interior of the spine the surgeon enters the spinal canal at the sacral hiatus. The entrance is created using a needle, but the instruments used during the operation require a larger diameter entrance than a needle can provide. This is complicated by the fact that the bony sacral hiatus is hidden beneath the flesh of the back, out of view of the surgeon. There is a need in the industry to provide a way for a surgeon to increase the diameter of the opening in the sacral hiatus, while leaving the guide wire in place, reducing steps, reducing the possibility for errors, and saving time during the operation. Several patents disclose a surgical rasp, such as U.S. Pat. No. 5,342,365, does not disclose a way to leave the guide wire in place, or guide the surgeon to the correct location. U.S. Pat. No. 6,660,041 discloses a hollow rasp, but not a rasp that works together with a guide wire.

What is needed is a device that will allow a surgeon to leave a guide wire in place and use the wire to lead the instruments of the surgeon to the correct location within the patient, lowering the risk of complications during surgery, in particular, but not limited to, spinal surgery.

### SUMMARY

A device for enlarging the diameter of a hole within the spine while allowing the surgeon to leave a guide wire in place, and using the guide wire to control the movement of the device. Allowing the surgeon to leave the guide wire in place prevents multiple insertions and removals of the guide wire, while allowing the surgeon to benefit from its presence and avoid having to blindly search for the hole in the spine.

In one embodiment, a cannulated sacral introducer rasp is disclosed, comprising a solid body, the solid body having a front end and a back end, an abrasive surface, the abrasive surface covering a portion of the solid body and a longitudinal bore, the longitudinal bore starting near the front end and ending on a surface of the solid body.

In another embodiment, a means for increasing a diameter of a hole at a base of a spine is disclosed, comprising a means for being held, a means for increasing the diameter of the hole, and a means for guiding a surgeon to a correct location within the spine.

In another embodiment, a cannulated sacral introducer rasp is disclosed, including a body comprising a handle and a barbed section, the barbed section having a front tip and a shank, a starting hole, the starting hole near the front tip of the barbed section, an ending hole, the ending hole at a rear section of the shank of the barbed section, and a channel, the channel beginning at the starting hole, and ending at the ending hole.

### BRIEF DESCRIPTION OF THE DRAWINGS

The invention can be best understood by those having ordinary skill in the art by reference to the following detailed description when considered in conjunction with the accompanying drawings in which:

FIG. 1 is a perspective view of the surgery system incorporating the cushion support system and cannulated sacral introducer rasp.

FIGS. 2A and 2B illustrate the cushion support system.

FIGS. 3A through 3D illustrate the placement of individual cushions to create the cushion support system.

FIGS. 4A through 4C illustrate flexible placement of the cushions that make up the cushion support system.

FIG. 5 illustrates a bottom view of the leg isolation and tool support cushion.

FIG. 6 illustrates a top view of the pelvic cushion.

FIG. 7 illustrates a prior art rasp.

FIG. 8 illustrates a side view of the cannulated sacral introducer rasp.

FIGS. 9A and 9B illustrate the cannulated sacral introducer rasp in use.

FIG. 10 illustrates a prior art surgical laser.

FIG. 11 illustrates a pair of prior art surgical lasers.

FIG. 12 illustrates the multiple wavelength surgical laser system.

FIG. 13 illustrates a block diagram of a prior art surgical laser.

FIG. 14 illustrates a block diagram for the multiple wavelength surgical laser system.

FIG. 15 illustrates a graph of absorption by materials of ranges of wavelengths of light.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures. The examples below do not purport to represent all potential examples or embodiments of the invention, with many other potential examples possible by one skilled in the arts.

As described above, less invasive surgical methods and devices for treating spinal stenosis and other back problems often utilize a laser to remove the infringing tissue. “Epiduroscopy” by G. Schültze describes such methods of performing spinal endoscopy using lasers. In this, G. Schültze describes methods for entering the epidural space, guiding a fiber optic probe into the epidural space with the help of a C-arm device and correcting various situations using a laser. In chapter 7.5, G. Schültze discusses the Epidural laser adhesiolysis, for example, using a 1064-nm Nd, YAG 1320-nm nd and a 940-nm laser for “coagulation of bleeding, rechanneling stenosis caused by tumors and destroying plaques in vessel walls.” In this, a fiber optic is introduced into the epidural space via a working channel of an epiduroscope under epiduroscope vision. A laser diode of from 1 watt to 25 watts fires a burst of energy through the fiber and onto the target tissue. G. Schültze describes that the light energy penetrates the tissues but is not significantly absorbed by the surrounding hemoglobin, melanin or water.

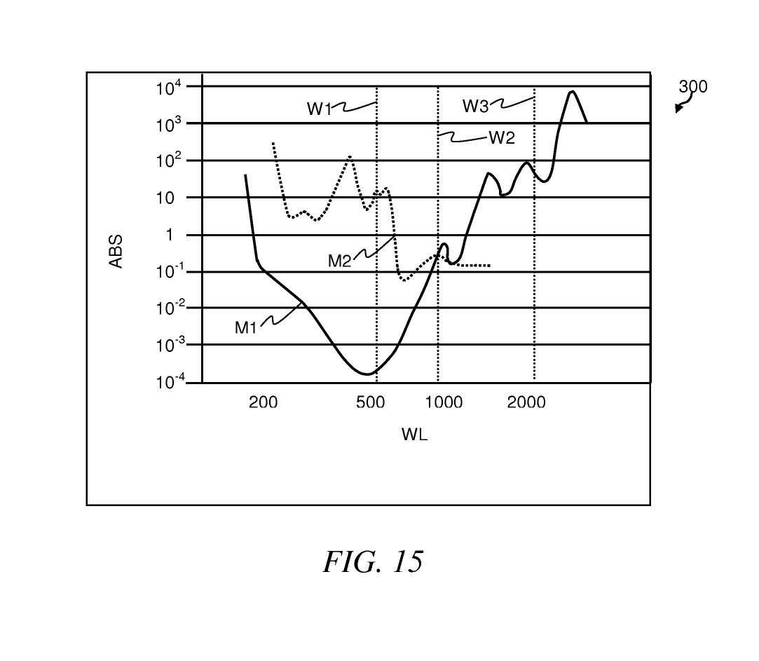

It is well known that different laser light frequencies are absorbed differently by different target materials. The described procedure in G. Schültze uses lasers with a wavelength of from around 940-nm to 1320-nm. These wavelengths are selected because they are well absorbed by both hemoglobin and water, which are both major components of cartilage and scar tissue. As shown in FIG. 15, light is well absorbed by hemoglobin up to approximately 1400 nm, while absorption by water becomes significant after 800 nm. From the point at which hemoglobin and water intersect on the absorption chart, approximately 980 nm, absorption by water increases significantly while the absorption by hemoglobin slowly decreases and becomes non-existent after 1400 nm. For this reason, different wavelengths of light energy are needed to remove tissue containing the target chromophore, or portion of the molecule responsible for the molecule's color. For example, a 532-nm (Green-light) has proven successful in vaporizing tissue containing large amounts of hemoglobin, such as enlarged prostate tissue, etc.

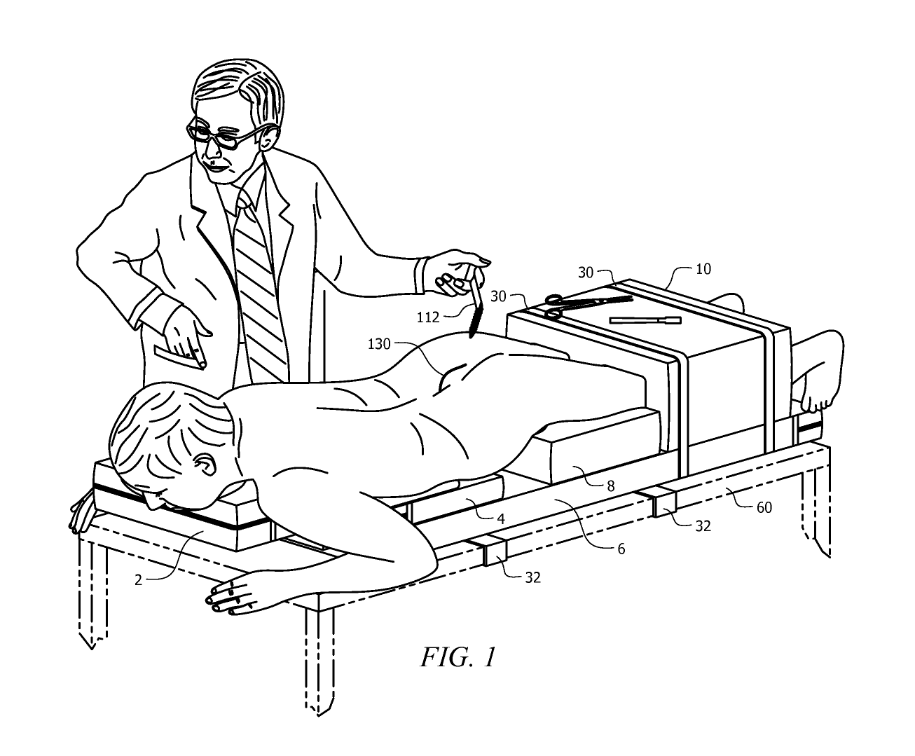

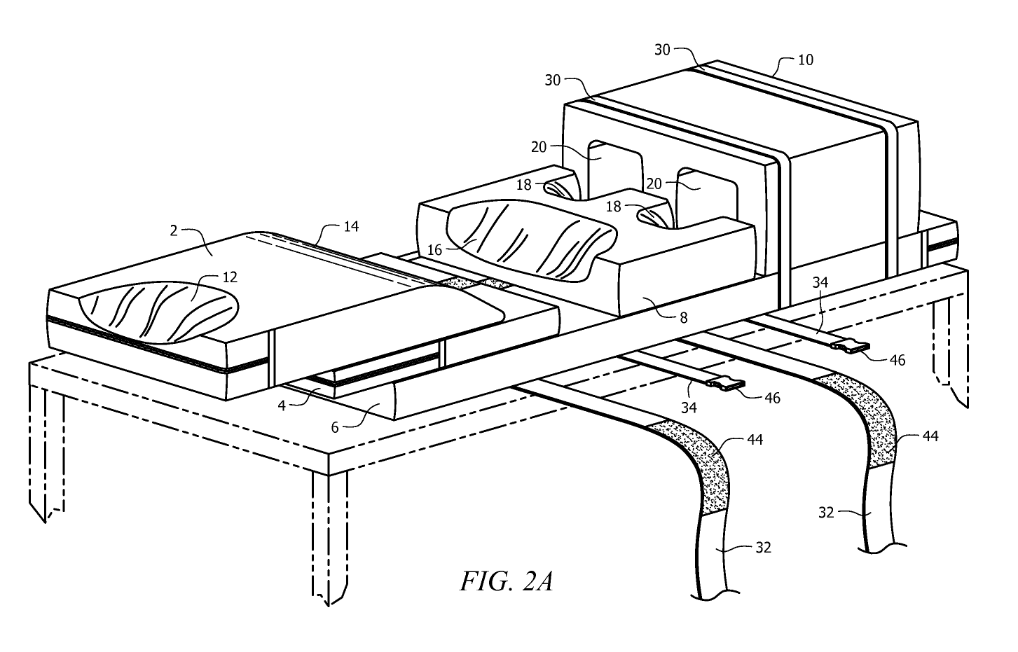

Referring to FIGS. 1, 2A, and 2B an exemplary cushion support system and cannulated sacral introducer rasp will be described. The cushion system comprises the chest support cushion 2, chest height adjustment cushion 4, main support cushion 6, pelvic cushion 8, and leg isolation and tool support cushion 10. The chest support cushion 2 includes a depression 12 for the patient's chin, and a slope 14 to support the angle of the chest. The shape of slope 14 varies for different embodiments to address the different shape of a man's chest as compared to a woman's chest. The depression 12 allows for airway access by an anesthesiologist. The cushion system comfortably positions the patient for spinal surgery. The chest support cushion 2 and chest height adjustment cushion 4 hold the chest and head, while the pelvic cushion 8 supports the pelvis, allowing the belly to hang. It is well known in the industry that many patients that have back issues are also obese. When a patient lies on their stomach, especially an obese patient, displacement of the abdomen creates higher fluid pressure in the spinal canal, particularly the fluid pressure in the epidural venous plexus surrounding the spinal nerves. Suspension of the abdomen by the disclosed cushion support system decreases this pressure, reduces the risk of side effects of surgery and provides improved access to the guide wire 130. The chest height adjustment cushion 4 is optional and is used to adjust the system for the size and shape of the patient. Whether or not the chest height adjustment cushion 4 is used, the top of the chest support cushion 2 is preferably higher than the pelvic cushion 8. This difference in height provides for having the chest and head lifted, allowing the abdomen and belly to hang, decreasing the venous pressure in the epidural veins in the spinal canal, as well as reducing the risk of retinal damage from pressure caused by use of the epiduroscope and fluid pressure infused to maintain the patency of the surgical field. The fluid helps to float the spinal sac and fat tissue away from the camera fiber optic distal lens and increase the visibility of the fiber optic scope during use of the high power light or laser. The cushion support system enables the abdominal viscera to hang freely, which creates a gravity dependent pooling of blood in the abdominal visceral blood vessels, with the most pronounced effects present in the venous system. The cushion support system creates negative pressure in the epidural veins and epidural capillaries via the connecting blood vessels. As a result, during the epiduroscopic surgical procedure the vessels are not engorged, and the risk of injury to these vessels is reduced, as well as the incidence of bleeding which obstructs the view the operative field. These benefits lower the risk of complications during back surgery.

It is anticipated that in other embodiments the effect of using the chest cushion is achieved by sloping the operating table 60 to place the level of the head above that of the pelvis. The pelvic cushion 8 has an abdominal depression 16 for a patient's belly and male genitalia, and two leg depressions 18, one for each of the patient's thighs. The leg isolation and tool support cushion 10 has two passageways 20, one for each of the patient's legs. The flat, table-top portion of the cushion 10 provides the physician a stable location for instruments. The main support cushion 6 has a plurality of straps 30/32, including straps 30 for holding the leg isolation and tool support cushion 10 in place, straps 32 for wrapping around either the patient or the operating table, and straps 34 for wrapping around the operating table. The straps removably connect in a multitude of ways such as by hook-and-loop fasteners 44, and/or buckles/snaps 46. The straps 34 keep the pad 6 removably affixed to the operating table 60.

The cushion system is best used with the head of the operating table elevated 30 degrees. This slope reduces fluid pressure in the spinal canal and reduces fluid pressure of the Cerebral Spinal Fluid (CSF), and further reduces the risk of retinal detachment by CSF fluid elevation.

In some examples, the cushion system is radiolucent, or substantially transparent to the passage of X-rays. In other embodiments the cushion system is substantially transparent to other types of signals, including those used in magnetic resonance imaging and ultrasonic imaging.

The cushions are constructed of any of a multitude of suitable materials as known in the industry. The inside of the cushion is preferably made from a supportive material such as closed-cell foam. Other inner materials are anticipated, including, but not limited to, open-cell foam, closed-cell foam, cushions of multiple material types (e.g., a stiff inner core and soft outer layer), natural and synthetic fillers, and all others as commonly known in the art. The outer covering of the cushion is preferable made from a water-resistant or water-proof fabric to facilitate cleaning. Other outer coverings are anticipated, including synthetic and natural fabrics, genuine and faux leather, and all others as commonly known in the art. In some embodiments, the cushions have an inner covering that is heat sealed to prevent any fluids from entering the foam. Such fluids could be present during the surgical process, or during cleaning.

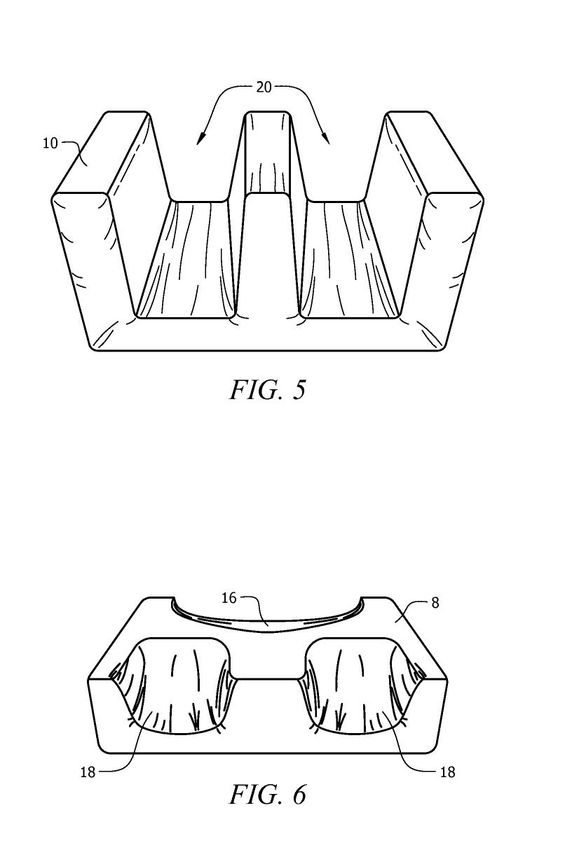

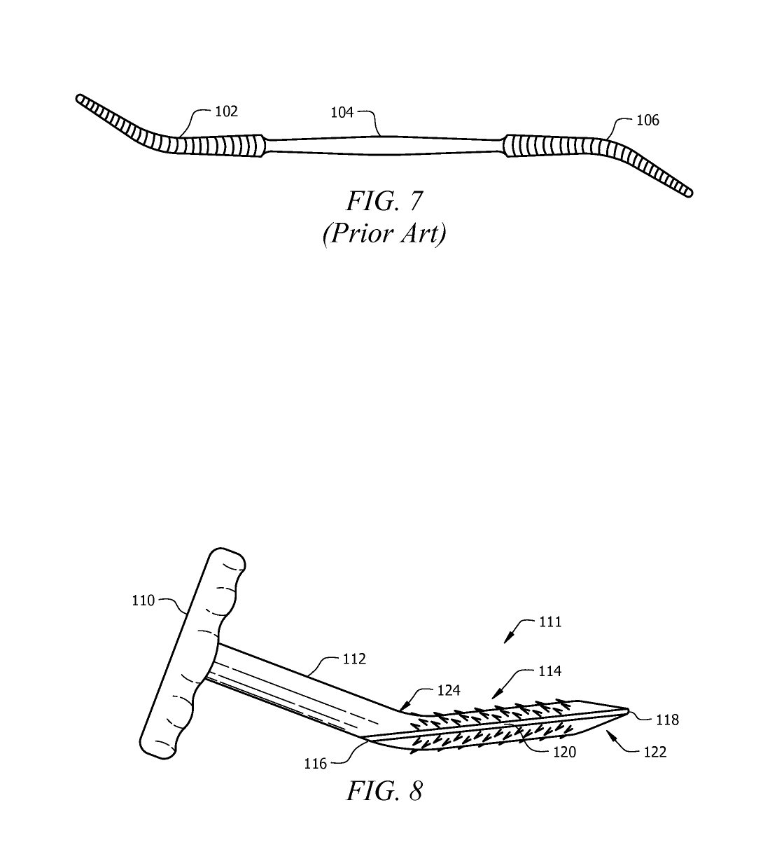

In FIG. 1, the surgeon is prepared to insert the cannulated sacral introducer rasp 112 (see FIGS. 8 and 9) into the spine while guided by the guide wire 130, creating better placement within the spine and fewer steps in surgery.

There exist many different means of removably affixing the cushions to each other, but in this example of the cushion support system, the cushions are held removably affixed to each other by hook-and-loop fasteners. Any means of temporarily affixing the cushions together will constitute removably affixing. Other methods of removably affixing cushions to each other, or any other surface, are anticipated, including hook-and-loop material, snaps, magnets, hooks, and all others as commonly known in the art. Although removably affixing is preferred, in some embodiments some or all of the cushions are permanently affixed to each other. In this example, the main support cushion 6 has a line of hook-and-loop fasteners 40 on one side. The hook-and-loop fasteners on main support cushion 6 interfaces with corresponding hook-and-loop fasteners on the bottom (not shown) of cushions 2 and 8. Also, in this example, the chest height adjustment cushion 4 has hook and loop fasteners on the top 42, and bottom (not shown) to connect to the cushions 2/6 above and below.

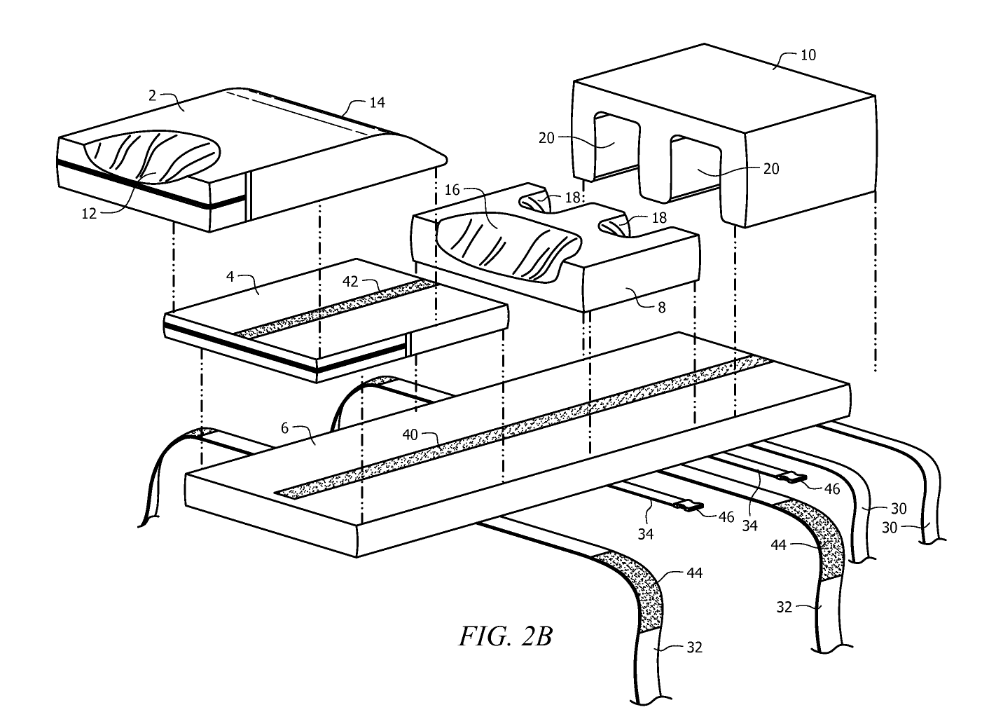

Referring to FIGS. 3A through 3D, the exemplary cushion support system will be further described. FIG. 3A is a side view of the main support cushion 6 and chest support cushion 2. FIG. 3B shows the addition of the chest height adjustment cushion 4 to raise the chest support cushion 2. FIG. 3C shows the addition of the pelvic cushion 8. FIG. 3D shows the addition of the leg isolation and tool support cushion 10, that is held by straps 30 in this example. Other methods of holding the tool support cushion 10 are anticipated, including hook-and-loop material, snaps, magnets, hooks, and all others as commonly known in the art.

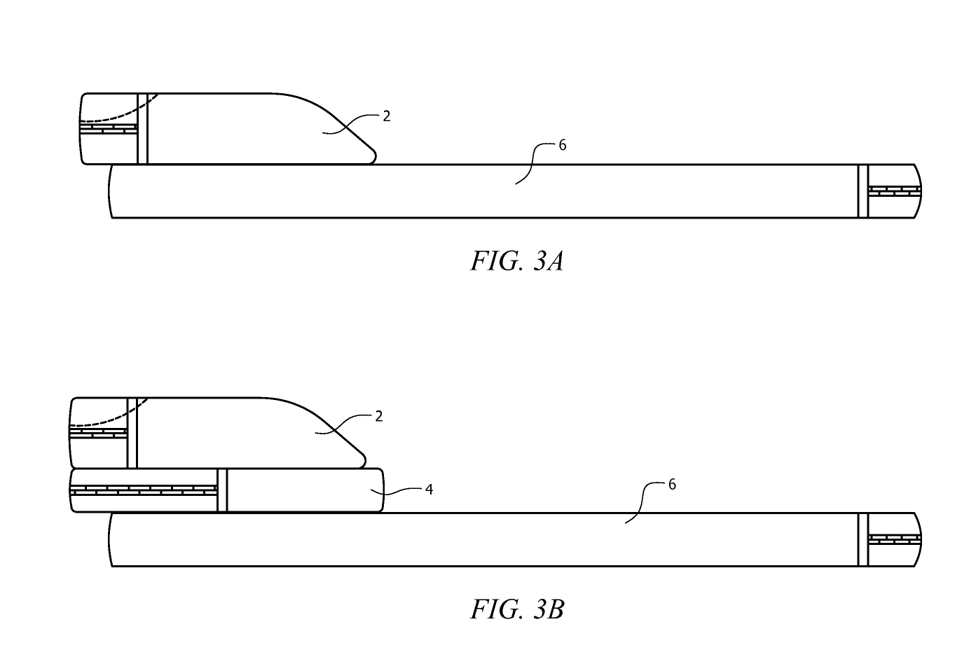

Referring to FIGS. 4A through 4D, the flexibility of the exemplary cushion support system will be described. FIG. 4A shows the cushions 2/4/6/8/10 positioned for an average sized patient. FIG. 4B shows the cushions 2/4/6/8/10 with closer positioning for a shorter patient. FIG. 4C shows the cushions 2/4/6/8/10 positioned for a taller patient.

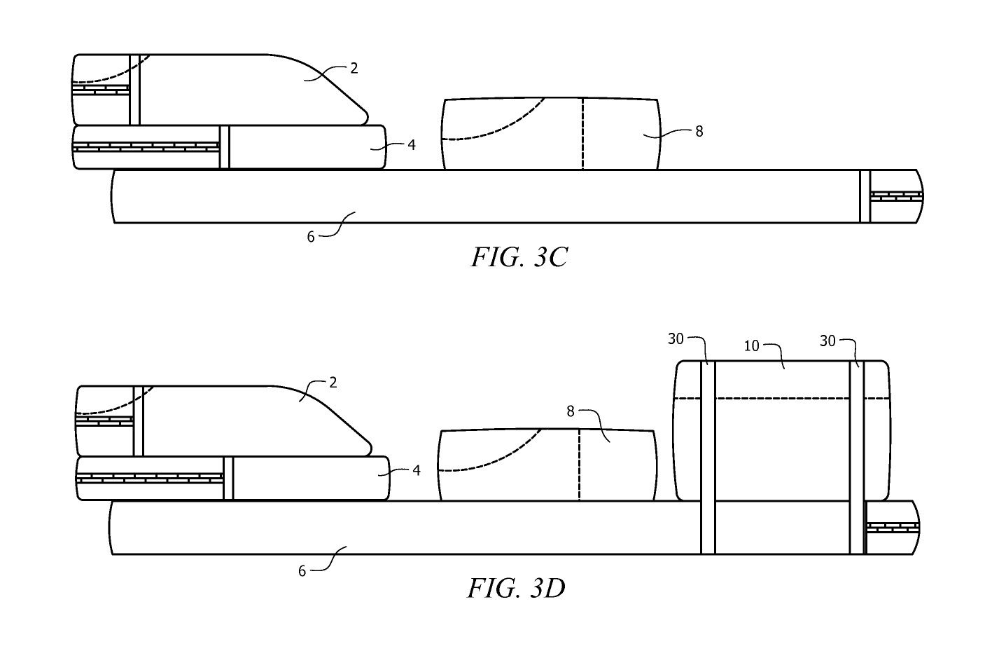

Referring to FIG. 5, the leg isolation and tool support cushion 10 will be described. FIG. 5 shows the bottom of cushion 10 with two passageways 20 for the patient's legs in an inverted position. When the cushion is upright, the large flat surface (visible in FIGS. 1, 2A and 2B) provides a working area for the physician, both for placing instruments that are needed during the procedure and creating a steady platform for hands and arms.

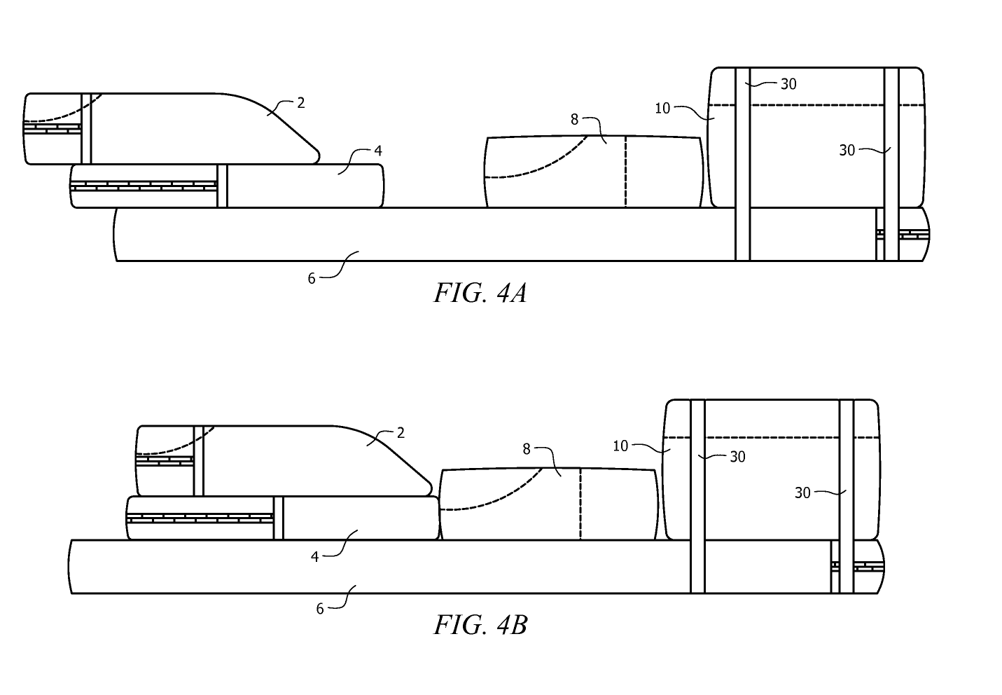

Referring to FIG. 6, the pelvic cushion 8 will be described. The top of the cushion has a first abdominal depression 16 and the two leg depressions 18 for the patient's thighs. The cushion is shaped to support the pelvic bones, with the depressions 16/18 in locations to minimize pressure on the patient's thighs and provide a location for the patient's abdomen to hang.

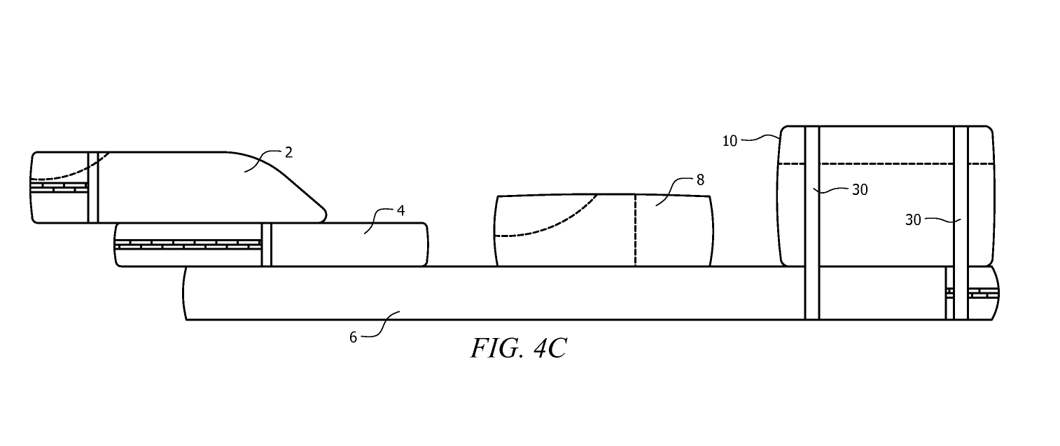

Referring to FIGS. 7 and 8, the prior art rasp, and new and improved rasp, will be described. In the prior art, many rasps are available. The example shown has two curved end sections 102 and 106 with teeth or ridges to remove material when rubbed along the surface. The ends are connected with a smooth section 104 where the rasp is typically held by the physician.

FIG. 8 shows the improved rasp 111. In this example, the cannulated sacral introducer rasp 111 consists of a T-shaped handle 110, a smooth section 112 followed by a barbed section 114, although other handle shapes and configurations are anticipated as known in the art. There is a bore or channel 120 passing preferably, though not required, through the axis of the barbed section. An entrance hole 118 and an exit hole 116 provide access to the bore 120. The entrance hole 118 is preferably near the front tip area 122 (the tip that is distal from the handle area) and the exit hole 116 is preferably near the rear shank area 124. The bore or channel 120 is preferably formed or drilled substantially through the center of the barbed section 114, starting near the front tip area 122 and ending near the rear shank area 124. Other hole 116/118 and bore/channel 120 locations and orientations are anticipated, including channels that are not situated through the center, channels that are not completely enclosed (e.g., a trough on one side of the barbed section), different hole locations, including locations at different sites on the handle 110, shank 112 or barbed area 114 are anticipated. In one embodiment, the holes 116 and 118 are conical openings to facilitate acceptance of a guide wire 130. In some embodiments, the holes 116/118 are of other shapes, including, but not limited to beveled edges, rounded edges, straight edges, and any other type of hole edge as commonly known in the art.

The holes 116/118 and bore 120 are preferably sized slightly larger than the guide wire 130, thereby allowing smooth movement of the guide wire 130 through the bore 120. Typically, the guide wire 130 has a circular cross-section and, therefore, the preferred bore 120 also has a circular cross-section, although any bore 120 cross sectional geometry is anticipated to match the cross-sectional geometry of the guide wire 130 such as oval, etc.

In this example of the cannulated sacral introducer rasp 111, the conical end section 122 is smooth. The middle section 114 is covered with an abrasive surface made of smaller triangular barbs. The portion between the middle section 114 and the rear shank area 124 (end section) is covered with another abrasive surface that has larger triangular shaped barbs. Other arrangements of abrasive surfaces are anticipated, including a barbed tip, barbs with shapes other than triangular, barbs along the rear shank area, and any other type of barbs or arrangement of barbs as commonly known in the art.

Referring to FIGS. 9A and 9B, a typical use of the cannulated sacral introducer rasp 111 will be described. In order to gain access to the spinal canal, an epidural Tuohey needle (not shown) is inserted into the spinal canal at the sacral hiatus 132. Following the insertion, dye is injected. Next, a guide wire 130 is passed through the needle. In the prior art, the guide wire is then removed and the prior art rasp 104/106, as shown in FIG. 7, is inserted blindly to enlarge the opening in the sacral hiatus 132. The prior art rasp 104/106 is then used to widen the hole created by the needle. In the prior art, because the guide wire must be removed prior to inserting the rasp, the physician is unable to verify the rasp is properly inserted. Additionally, in the prior art, the guide wire is later reinserted after the hole is widened, creating additional steps and increasing the risk of infection.

The cannulated sacral introducer rasp 111 has a channel 120 that runs through the rasp 111, allowing the rasp 111 to slide over the guide wire 130. The rasp is positioned at the entry to the sacrum 132, at the lower end of the lumbar vertebrae 134, without removal of the guide wire 130. The guide wire 130 remains in place during enlargement of the entry channel and, therefore, there is no need to remove the guide wire 130 and reinsert the guide wire 130 later. The rasp 111 is used, for example, to remove ligaments at the base of the spine for spinal penetration by instruments. The hole created by the rasp 111 also gives fluids an easy exit from the spine.

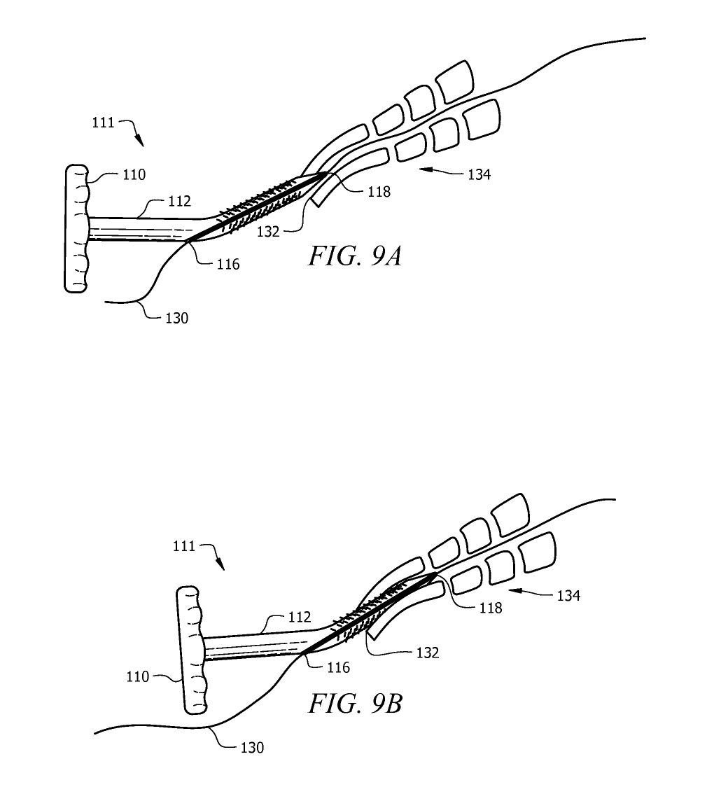

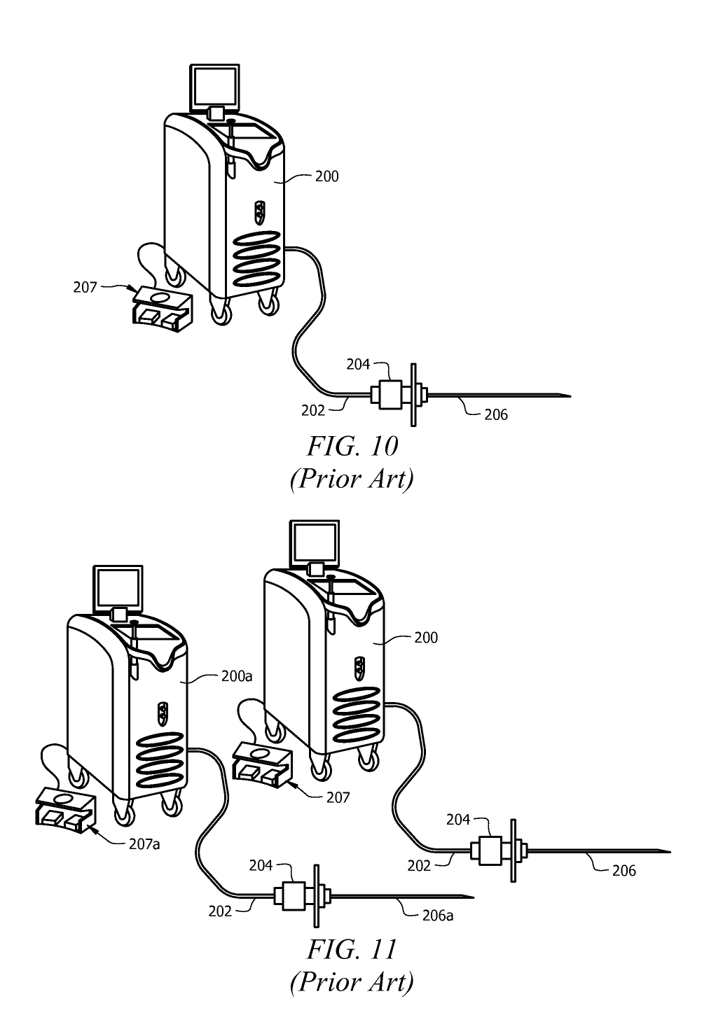

Referring to FIGS. 10 and 11, prior art surgical lasers are shown. Lasers have been used in the past for various types of surgery, including lower back surgery. Laser systems for such procedures are typified by the laser system shown in FIG. 10 consisting of a base system 200 that encloses the electronics used to produce a single wavelength laser beam, a cable 202 containing a fiber optic delivery bundle 206 having one or more individual fiber optic threads and a handle 204. The fiber optic delivery bundle 206 extends beyond the handle for insertion into the patient. The fiber optic delivery bundle directs a single wavelength laser beam at the target tissue within the patient. It is known in the art that different tissues react differently to exposure of high-intensity light radiation of different wavelengths. For example, in “Epiduroscopy” by G. Schültze, methods of performing spinal endoscopy using lasers are described. In this, G. Schültze describes methods for entering the epidural space, guiding a fiber optic probe into the epidural space with the help of a C-arm device and correcting various situations using the laser. In chapter 7.5, G. Schültze discusses the Epidural laser adhesiolysis, for example, using a 1064-nm Nd laser, a YAG 1320-nm Nd laser, and a 940-nm laser for “coagulation of bleeding, rechanneling stenosis caused by tumors and destroying plaques in vessel walls.” In this procedure, a fiber optic cable is introduced into the epidural space via a working channel of an epiduroscope under epiduroscope vision. A laser diode of from 1 watt to 25 watts fires a burst of energy of the specific wavelength through the fiber and onto the target tissue. G. Schültze describes that the light energy penetrates the tissues but is not significantly absorbed by the surrounding hemoglobin, melanin or water (see FIG. 17). The prior art laser 200 of FIG. 10, having a single wavelength output is very useful in removing a single type of tissue.

In these procedures, when multiple wavelengths of laser are needed to remove different types of tissue (e.g. hydrated bulging disc tissue as opposed to desiccated, degenerated disc tissue), multiple laser systems 200 of the prior art were used as shown in FIG. 11. During the prior art procedures, the fiber optic bundle 206 from the first laser system 200 is inserted into the epidural area of the patient and the laser system 200 is triggered through a foot switch 207 to radiate the first type of tissue (e.g. ligament tissue, or well hydrated bulging disc tissue) with a prescribed power of the first wavelength of light, thereby vaporizing that first type of tissue. Now, if a second type of tissue is encountered (e.g. dessicated, degenerated disc tissue), the first fiber optic bundle 206 is pulled out from the epidural area and a second fiber optic bundle 206A is inserted. The second fiber optic bundle 206A is interfaced to a second laser system 200A (as shown in FIG. 11) which emits a different wavelength of laser light than that of the first laser system 200. Again, the laser system 200A is triggered through a foot switch 207A to radiate the second type of tissue (e.g. dessicated, degenerated disc tissue) with a prescribed power of the second wavelength of light, thereby vaporizing that second type of tissue. During a single operation, it is often required to repeat these steps as different types of tissue are exposed and need to be removed.

In addition to requiring extra steps of removal and insertion by the surgeon into and out of the patient, increasing the opportunity for infection, having two or more laser systems 200/200A increases cost because many of the components of the first laser system 200 are duplicated in the second and subsequent laser systems 200A.

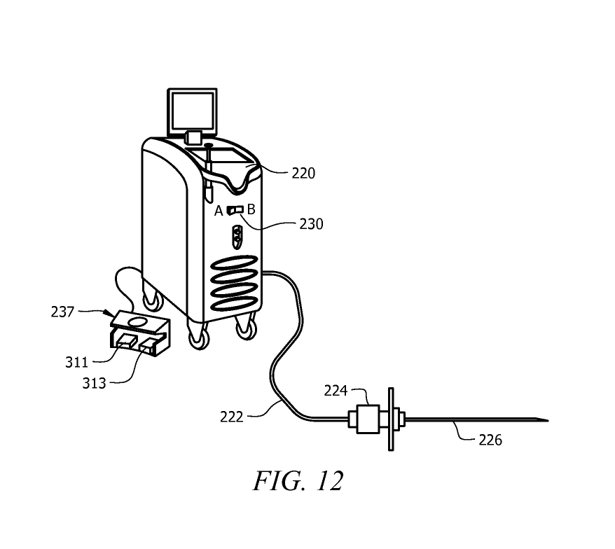

Referring to FIG. 12, the multiple wavelength surgical laser 220 is shown. The multiple wavelength surgical system 220, shown in an exemplary enclosure, emits two or more different wavelengths of light (e.g. laser light) radiation through a single cable 222 having one or more fiber optics within a fiber optic bundle 226 that deliver the laser energies to the abnormal tissue within the patient. Although shown having a specific handle 224 and cable system 222/226, any known delivery of the two or more wavelengths of laser radiation is anticipated.

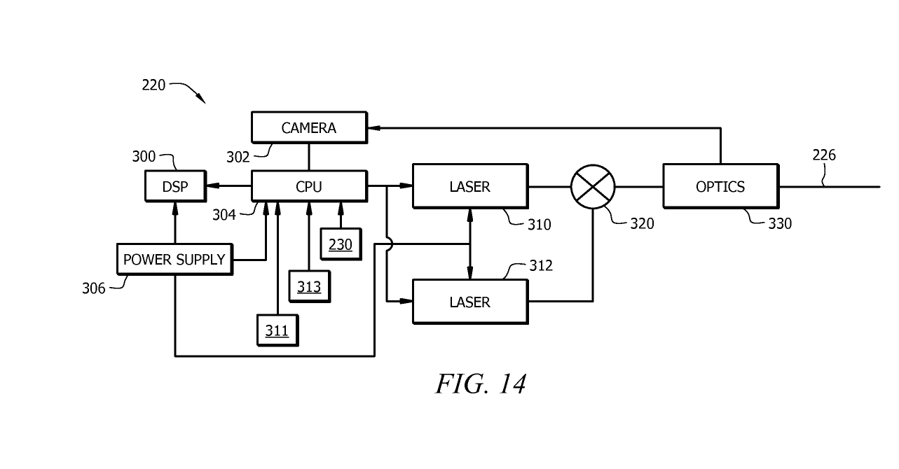

A light emitting device 310/312 (see FIG. 14) is any device that provides light, the light being able to be focused into a beam and the light sufficient (e.g. high power) to affect targeted tissue. An example of such a device is a laser 310/312, but there is no limitation that lasers are the only allowable source of energy or light energy. Any light emitting device can be substituted, or devices that provide other sources of focused energy, including energy not classified as light.

In order to be useful, the light emitting device needs to emit sufficient energy as to affect the targeted tissue, referred to in this description as “high-power.” Using a laser as an example, existing laser light emitting devices have power outputs that range from a 1 mW laser pointer to a 100 kW or greater laser used in weaponry and research applications. To be effective for surgical use, a laser 310/312 (or other light output device) needs to produce sufficient power output as to affect the target tissue while not damaging surrounding tissue or other parts of the patient's body. Light power outputs in the range of 1-25 watts have been shown useful in affecting many types of unwanted mammalian tissue. The interaction between the light source and tissue will vary depending upon the type of light utilized, the wavelength, the light generator source, the power level, pulsed vs. non-pulsed deliver of the light, and the energy field created (i.e., direct surface contact with light, or heating of surrounding tissue with formation of a steam bubble with subsequent tissue vaporization).

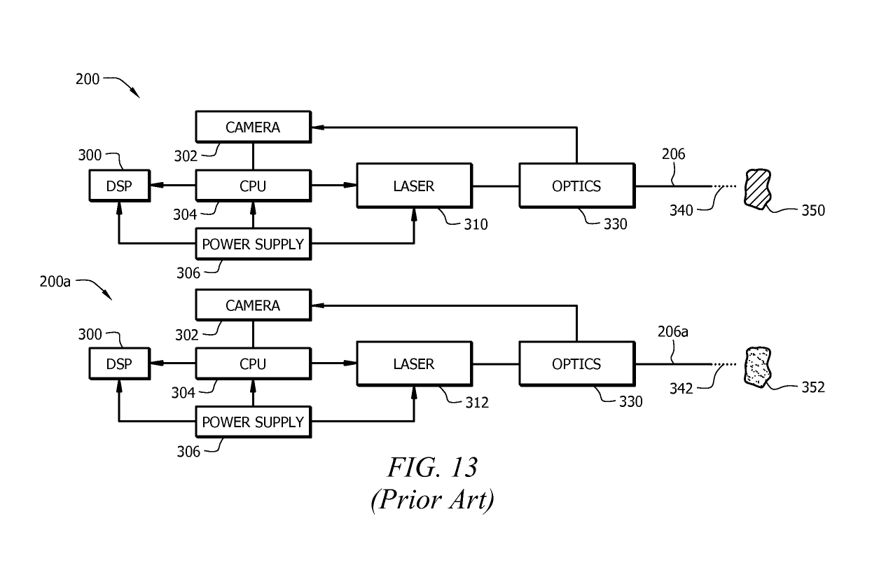

Many existing surgical laser systems 200/200A provide for controls to adjust the output power of the laser. It is anticipated that, in some embodiments, the multiple wavelength surgical laser 220 also has an adjustment to control the power output of each individual source 310/312 (see FIG. 13) of laser radiation, or to simultaneously control the individual sources 310/312 by application of a common ratio of power between the sources 310/312. All types of on/off and power setting controls are anticipated, including foot switches, voice control, a pressure switch, eye recognition, computer control, etc.

Instead of alternating between fiber optic bundles 206/206A of the prior art, a single fiber optic bundle 226 delivers multiple wavelengths of laser radiation to the target tissue. The wavelength of laser radiation passing through the fiber optic bundle 226 is controlled by switching from one laser source 310/312 to a different laser source 310/312 by, for example, a selector switch 230 or different foot switches 311/313 within a foot pedal 237 or any other mechanism known in the art. In some embodiments the wavelengths are delivered simultaneously at a fixed or variable ratio of power, as desired and set by the laser operator.

Referring to FIG. 13, a block diagram of a prior art surgical laser is shown. In the prior art, two different laser systems 200 were employed, each duplicating most or all of the components of the other and each delivering their wavelength of laser radiation through one or more fiber optics within a fiber bundle 206. Each of the prior art laser systems 200/200A had its own power supply 306, display 300, processor 304, optics 330 and light (laser) radiation generator 310/312 such as a laser diode 310/312 or any other source of laser radiation. In the example of the prior art shown, the first laser systems 200 has a first laser 310 emitting a first wavelength of laser radiation that is best for use with vaporizing a first type of tissue 350 (e.g. ligament, or well hydrated bulging disc tissue). The second laser systems 200A has a second laser emitting a second wavelength of laser radiation that is best for use with vaporizing a second type of tissue 352 (e.g. dessicated, degenerated disc tissue).

As stated before, the systems of the prior art required the surgeon to pull out one laser fiber bundle 206 and insert another laser fiber bundle 206A when operating on a different type of tissue. FIG. 13 shows the duplication of components found in the prior art such as the display 300, the camera 302, the processor 304, the power supply 306, the optics 330 and the fiber bundle 206. This duplication is not present in the system shown in FIG. 14.

Referring to FIG. 14, a block diagram for the new multiple wavelength surgical system 220 is shown. The multiple wavelength surgical systems 220 has a power supply 306, a display 300, a processor 304, optics 330 and two or more light (e.g. laser) radiation generators 310/312 such as a laser diode 310/312 or any other source of directed light radiation. Each light radiation generator 310/312 delivers their respective wavelength of light radiation through one or more fiber optics within a single fiber bundle 226. In use, the epidural space is opened, in some embodiments using a rasp, then the fiber optics are guided into the epidural space, preferably with the help of a C-arm device. One or multiple sources of light 310/312 are fired as needed to correct various situations such as to remove a first type of tissue (e.g. ligament tissue, or well hydrated bulging disc tissue) with a prescribed power of the first wavelength of light, thereby vaporizing that first type of tissue or to remove a second type of tissue (e.g. dessicated, degenerated disc tissue) with a prescribed power of the second wavelength of light, thereby vaporizing that first type of tissue, etc. In the example shown, laser radiation from each of the laser radiation generator 310/312 are mixed or switched into the fiber optic bundle 226 by a light mixer 320 or light switch 320 as known in the industry. Alternately, the laser radiation from each of the laser radiation generator 310/312 is interfaced to its own, dedicated fiber optic within the single fiber optic bundle 226. Any number of laser radiation generators 310/312 is anticipated. Multiple wavelengths can be delivered independently or simultaneously through the fiber optic. Again, the system is not limited to any particular source of light and lasers 310/312 are examples of such sources of light.

The first laser radiation source 310 emits a first wavelength of laser radiation that is best for use with vaporizing a first type of tissue 350 (e.g. ligament or scar tissue). The second laser radiation source 312 emits a second wavelength of laser radiation that is best for use with vaporizing a second type of tissue 352 (e.g. disc tissue). In embodiments with three wavelengths, a third laser radiation source (not shown) emits a third wavelength of laser radiation that is best for use with vaporizing a third type of tissue (not shown), and so forth. Again, any number of laser radiation sources 310/312 is anticipated. Any number of wavelengths can be delivered independently or simultaneously through the fiber optic.

In some embodiments, the laser radiation from the two or more laser sources 310/312 is either combined or switched by a light mixer/switch/multiplexor 320 and directed into one or more fiber optic fibers 226 through an optical system 330 as known in the industry. In other embodiments, laser radiation from each of the two or more laser sources 310/312 is directed into its own set of one or more fiber optic fibers 226 through an optical system 330 as known in the industry.

To control which of the laser radiation generator 310/312 is selected and subsequently excited to deliver its respective wavelength of laser radiation, a control 230/318 is provided such as a selector switch 230 or multiple floor switches 311/313, etc. For example, when the surgeon needs the first wavelength of laser radiation, the surgeon moves the selector switch 230 to a first position then initiates emission of the laser energy by, for example, pressing the foot switch 311/313 with a foot. All types of control are anticipated, including foot switches, voice control, pressure switches, eye recognition, computer control, etc. When the surgeon needs the second wavelength of laser radiation, the surgeon moves the selector switch 230 to a second position, then initiates emission of the laser energy by, for example, pressing the foot switch 311/313 with a foot. In another embodiment, when the surgeon needs the first wavelength of laser radiation, the surgeon initiates emission of the laser energy by pressing a first switch 311 the foot switch 237 with a foot. When the surgeon needs the second wavelength of laser radiation, the surgeon initiates emission of the laser energy by, for example, pressing a second switch 313 of the foot switch 237 with a foot. Many ways are known to control the emission of the laser energy, all of which are included here within. In embodiments in which multiple wavelengths of laser energy are concurrently delivered, one or more switches (not shown for brevity purposes) or foot switches (not shown for brevity purposes) are provided for concurrently delivering two or more of the wavelengths of laser energy at the same time. The individual sources 310/312 are individually or simultaneously controlled by application of a common ratio of power between the sources 310/312.

Referring to FIG. 15, a graph 300 of absorption by materials of ranges of wavelengths of light is shown. This graph illustrates absorption of two different materials (M1 and M2). As shown by the graph 300, a first material containing water M1 (e.g. H₂O) readily absorbs laser energies above around 800 nano meters, but poorly absorbs laser energies between around 200 nano meters and 800 nano meters. A second material containing Hemoglobin M2 (e.g. H₂O₂) readily absorbs laser energies between 200 and 600 nano meters, but poorly absorbs laser energies above 600 nano meters. Therefore, laser energy from a 532 nano meter laser W1 will be highly absorbed by tissue containing Hemoglobin but will barely be absorbed by tissue containing water, while laser energy from a 982 nano meter laser W2 will be absorbed by tissue containing either water or hemoglobin, and laser energy from a 2100 nano meter laser W3 will be absorbed by tissue containing water but poorly absorbed by tissue containing hemoglobin. This is why different lasers are useful for vaporizing different classes of tissue.

While the application addresses the system, method, and device in terms of the use of lasers to produce light, there is no limitation that lasers are the only allowable source of energy or light energy. Any light emitting device can be substituted, or other sources of focused energy, including energy not classified as light, may be used in the same manner.

Additionally, the application addresses specific frequencies as exemplary due to the commercial availability of certain laser light frequencies. It is anticipated that as lasers become commercially available in other frequencies of light that they will be used within the system, method, and device to accomplish tissue removal.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A cannulated sacral introducer rasp comprising:
a solid body, the solid body having a front end and a back end;
an abrasive surface, the abrasive surface covering a portion of the solid body; and
a longitudinal bore, the longitudinal bore starting near the front end and ending on a surface of the solid body.

2. The cannulated sacral introducer rasp of claim 1,
wherein the longitudinal bore is of a size that allows a guide wire to pass freely through.

3. The cannulated sacral introducer rasp of claim 1, wherein the longitudinal bore passes through a center of the solid body.

4. The cannulated sacral introducer rasp of claim 1, further comprising:
a handle, the handle attached to the back end of the solid body.

5. The cannulated sacral introducer rasp of claim 1, wherein the abrasive surface is composed of barbs.

6. The cannulated sacral introducer rasp of claim 5, wherein the barbs are divided into:
an end section, the end section covered in barbs of a large size;
a middle section, the middle section covered in barbs of a small size; and
a tip section, the tip section absent of barbs.

7. The cannulated sacral introducer rasp of claim 1 further comprising:
a guide wire, the guide wire strung through the bore.

8. A means for increasing a diameter of a hole at a base of a spine comprising:
a means for being held;
a means for increasing the diameter, of the hole; and
a means for guiding a surgeon to a correct location within the spine.

9. The means for increasing a diameter of a hole at a base of a spine of claim 8, whereas the means for increasing the diameter is comprised of an abrasive section, the abrasive section having a front tip and a rear shank, the means for increasing the diameter interfaced to the means for being held.

10. The means for increasing a diameter of a hole at a base of a spine of claim 8, wherein the means for guiding a surgeon is comprised of a channel through the means for increasing the diameter though which a guide wire is passed.

11. The means for increasing a diameter of a hole at a base of a spine of claim 9, wherein the abrasive section is comprised of barbs.

12. The means for increasing a diameter of a hole at a base of a spine of claim 8, wherein the means for increasing the diameter is comprised of a solid body, the solid body having a channel, the channel passing through the solid body such that a guide wire may pass freely through.

13. A cannulated sacral introducer rasp comprising:
a body comprising a handle and a barbed section, the barbed section having a front tip and a shank;
a starting hole, the starting hole near the front tip of the barbed section;
an ending hole, the ending hole at a rear section of the shank of the barbed section; and
a channel, the channel beginning at the starting hole, and ending at the ending hole.

14. The cannulated sacral introducer rasp of claim 13, further comprising:
a connecting bar between the barbed section and the handle.

15. The cannulated sacral introducer rasp of claim 13, further comprising:
a series of barbs on an outside surface of the barbed section.

16. The cannulated sacral introducer rasp of claim 13, wherein the barbed section comprises:
an end section, the end section located towards the handle, the end section covered in barbs of a large size;
a middle section, the middle section located central to the barbed section, the middle section covered in barbs of a small size; and
a tip section, the tip section located towards the front tip, the tip section having no barbs.

17. The cannulated sacral introducer rasp of claim 13, wherein:
the starting hole is conical such that insertion of a guide wire is simplified; and
the ending hole is conical such that insertion of the guide wire is simplified.

18. The cannulated sacral introducer rasp of claim 13 further comprising:
a guide wire, the guide wire passing through the starting hole, continuing through the channel, and exiting through the ending hole.
